(19) United States
(12) Patent Application Publication
(10) Pub. No.: US 2026/0191511 A1
(43) Pub. Date: Jul. 9, 2026

(54) ULTRASOUND-BASED BLADDER SCANNER SYSTEM AND A BLADDER VOLUME MEASUREMENT METHOD IN THE SYSTEM

(71) Applicant: MCUBE TECHNOLOGY CO., LTD., Seoul (KR)

(72) Inventors: Soo Yeol LEE, Seongnam-si (KR); Hyun Hwa CHO, Hanam-si (KR); Jae Joon SUH, Suwon-si (KR); Moon Chang HUR, Gimpo-si (KR); Dae Hong LEE, Seoul (KR)

(21) Appl. No.: 19/558,240

(22) Filed: Mar. 5, 2026

Related U.S. Application Data
(63) Continuation-in-part of application No. PCT/KR2023/014572, filed on Sep. 22, 2023.

(30) Foreign Application Priority Data
Sep. 18, 2023 (KR) 10-2023-0123898

(51) Int. Cl. A61B 8/00; A61B 8/08; G06T 7/62

(52) U.S. Cl. CPC A61B 8/5223; A61B 8/085; A61B 8/461; G06T 7/62; G06T 2207/10132; G06T 2207/20076; G06T 2207/20081; G06T 2207/30084

(57) ABSTRACT

Provided is an ultrasound-based bladder scanner system and a bladder volume measurement method using the same. A processor of the bladder scanner system is configured to infer, using a pre-trained deep learning network, a probability value indicating whether each unit region constituting an ultrasound image is included in a region of interest. The processor determines a control parameter for each ultrasound image based on statistical characteristics of a probability distribution of the ultrasound image, generates an adaptive probability transformation function corresponding to each ultrasound image using the control parameter, converts the probability value of each unit region into a weight using the adaptive probability transformation function, and extracts information regarding the region of interest using the weights.

This invention was developed with support from the Korea Medical Device Development Fund, funded by the Ministry of Science and ICT, the Ministry of Trade, Industry and Energy, the Ministry of Health and Welfare, and the Ministry of Food and Drug Safety of the Republic of Korea under Project Nos. 1711194192 and RS-2020-KD000043.

### TECHNICAL FIELD

The present invention relates to an ultrasound-based bladder scanner system and a bladder volume measurement method performed by the system. In particular, the ultrasound-based bladder scanner system is configured to infer probability values indicating whether unit regions of an ultrasound image correspond to a region of interest by using a pre-trained region-of-interest extraction model based on a deep learning network, to generate an adaptive probability transformation function having control parameters adaptively determined based on statistical characteristics of a probability distribution of the ultrasound image, to convert the probability values into weights by using the adaptive probability transformation function, and to extract information on the region of interest by using the weights.

### BACKGROUND ART

In general, an ultrasound system is a system for examining an internal state of an object. The ultrasound system transmits an ultrasound signal to an object to be examined by means of a piezoelectric effect of a probe, i.e., a transducer, receives an ultrasound signal reflected and returned from a discontinuous surface of the object, converts the received ultrasound signal into an electrical signal, and outputs the electrical signal to a predetermined imaging device. Such ultrasound systems are widely used in medical diagnosis, non-destructive testing, underwater exploration, and the like. In particular, by extracting a region of interest, such as a specific organ or lesion, from a medical image acquired using the ultrasound system and measuring a size of the extracted region of interest, a technique for determining or diagnosing a condition of a patient has been widely used.

Meanwhile, in examinations for bladder abnormalities or voiding dysfunction, measuring the amount of urine in the bladder is used as an essential factor. In addition, in order to prevent urinary retention that may occur after surgery, the amount of urine in the bladder is measured prior to urination using a catheter, and in voiding training, the amount of urine in the bladder is also measured and used as a guideline. The amount of urine filled in the bladder is equal to the volume of the bladder. Accordingly, the amount of urine in the bladder may be measured by measuring the volume of the bladder. Further, an ultrasound scanner may be used to measure the volume of the bladder.

As described above, the ultrasound scanner for measuring the amount of urine in the bladder acquires ultrasound signals corresponding to fan-shaped scan planes including the bladder, obtains two-dimensional or three-dimensional ultrasound images from the ultrasound signals for the scan planes, extracts a bladder region, which is a region of interest, from the ultrasound images, calculates an area or a volume of the bladder using information on the extracted bladder regions, and estimates the amount of urine in the bladder based thereon. FIG. 1 illustrates an example of a bladder volume measurement system using an ultrasound scanner, and FIG. 2 illustrates an example of a two-dimensional ultrasound image obtained using the bladder volume measurement system of FIG. 1.

However, since a dedicated ultrasound scanner for bladder volume measurement is manufactured in a small-sized or portable form to be easily movable, ultrasound images obtained by the dedicated ultrasound scanner generally have a low resolution. Accordingly, due to the low resolution of images acquired by the dedicated ultrasound scanner for bladder volume measurement, pixels or voxels have a relatively large size. Due to such characteristics, not only does a partial volume effect occur in the ultrasound images, but also boundaries of a region of interest or lesions become ambiguous. In addition, noise and artifacts generated by the ultrasound scanner for acquiring medical images frequently occur. Accordingly, there is a problem in that it is generally difficult to accurately extract a region of interest from a low-resolution ultrasound medical image. Furthermore, due to such problems, it also becomes difficult to accurately measure an area or a volume of the bladder from the region of interest extracted from the ultrasound image.

Meanwhile, various extraction algorithms have been proposed for extracting a bladder region, which is a region of interest, from two-dimensional or three-dimensional ultrasound images. In the related art, methods for identifying and extracting a region of interest by analyzing a magnitude or intensity of an ultrasound signal have been used. More recently, techniques have been proposed for automatically extracting a region of interest, such as a bladder region, from an ultrasound image by using an artificial intelligence model or a neural network model based on machine learning technology.

U.S. Pat. No. 12,217,445, entitled “Probability Map-Based Ultrasound Scanning”, discloses a technique in which probability information associated with a region of interest is generated from an ultrasound image by using a machine learning algorithm, and the region of interest is extracted based on the probability information. In particular, the disclosed machine learning algorithm infers probability values indicating whether each pixel or each preset unit region of the ultrasound image belongs to the region of interest, and the inferred probability values are binarized based on a predetermined threshold to classify the pixels or unit regions, thereby extracting the region of interest.

However, in a binarization-based approach, probability values are forcibly converted into binary values according to a threshold, which may cause loss of probabilistic information inherently contained in the probability map. In addition, when the threshold is fixed or not adaptively determined in consideration of the statistical characteristics of the probability distribution of a given ultrasound image, extraction accuracy may vary depending on imaging conditions, signal noise, or patient-specific factors. Accordingly, there remains a need for an improved technique capable of utilizing probability information in a more flexible and adaptive manner to enhance the stability and accuracy of region-of-interest extraction.

In FIG. 3, (a) is a two-dimensional ultrasound image 810 acquired using a bladder scanner system, (b) is an image 820 generated by reconstructing probability values output for each pixel of the two-dimensional ultrasound image of (a) using a machine learning algorithm, (c) is an enlarged view of a portion 830 of (b), and (d) is a graph 840 showing the probability values corresponding to (c). As shown in (d) of FIG. 3, each output probability value has a value between 0.0 and 1.0, and when the probability value is 1.0, it indicates that the corresponding pixel is classified as belonging to the bladder with the highest confidence level provided by the model.

In the conventional technique, in order to obtain an area or a volume of a bladder, which is a region of interest, from an ultrasound image, a binarization method for detecting a boundary from an image such as that shown in FIG. 4A is used. The binarization method separates a background region and a region of interest using a preset threshold.

FIG. 4A illustrates a two-dimensional ultrasound image. FIG. 4B illustrates an image reconstructed from probability values output by an extraction algorithm. FIG. 4C illustrates an image obtained by binarizing the probability values with a threshold set to 0.5. FIG. 4D illustrates an image obtained by binarizing the probability values with a threshold set to 0.4. In the case of FIG. 4C, the number of pixels having probability values equal to or greater than the threshold is 2,554, whereas in the case of FIG. 4D, the number of pixels having probability values equal to or greater than the threshold is 2,661. As illustrated in FIGS. 4A to 4D, the boundary position of the region of interest varies depending on the threshold value, and the extracted region of interest also varies accordingly. As a result, an area or volume of the region of interest calculated based on the extracted region likewise varies. That is, as the threshold value decreases, a size of the bladder, which is the region of interest, increases in proportion to a decrease in the threshold value.

FIG. 5 illustrates a graph exemplarily showing a change in probability values according to a scan depth along a single scan line in a bladder volume measurement system using an ultrasound scanner according to the related art. Referring to FIG. 5, in region I, since the probability values are close to 0, the corresponding region may be inferred with high confidence as being outside the bladder. In contrast, in region III, since the probability values are close to 1, the corresponding region may be inferred with high confidence as being inside the bladder. However, in region II, the probability values are distributed between 0 and 1, and thus it may be inferred that the corresponding region corresponds to a bladder boundary region. Nevertheless, because a width of the boundary region is relatively large, it is difficult to clearly specify a starting point of the bladder. The shape and width of such a boundary region may vary depending on various parameters, including an angle at which an ultrasound scan plane and a scan line intersect the bladder, as well as a shape, position, and size of the bladder.

Accordingly, a method of determining a bladder region by applying a single threshold value to a probability map and binarizing the probability map has limitations in terms of reliability. That is, when a relatively low threshold value t₁ or a relatively high threshold value t₂ is applied, there is a risk that a size of the extracted bladder region may vary excessively.

In addition, a manner in which a size of the bladder, which is the region of interest, varies according to a change in the threshold value may differ depending on a size or shape of the bladder, a position of the bladder within the abdomen, and a condition of digestive organs surrounding the bladder. Furthermore, in the case of female subjects, the variation pattern may also differ due to anatomical factors such as a condition of the uterus or the presence of cysts. Accordingly, a method of extracting a region of interest from an ultrasound image by applying a single threshold value does not readily ensure stable extraction of the region of interest under various patient conditions and measurement conditions.

As described above, when a binarization method is used to extract a region of interest from an ultrasound image, an area or volume of the region of interest varies sensitively depending on a threshold value used for binarization. Accordingly, the threshold value has a substantial impact on reliability of a final measurement result.

Accordingly, the present invention proposes a method of extracting information on a region of interest from an ultrasound image more accurately and reliably by using probability values corresponding to unit regions of the ultrasound image, without relying on threshold-based binarization or segmentation.

### SUMMARY OF THE INVENTION

In order to address the above-described issues, an object of the present invention is to provide an ultrasound-based bladder scanner system and a bladder volume measurement method performed by the system, configured to extract probability values corresponding to pixels or unit regions of an ultrasound image by applying a region-of-interest extraction algorithm to the ultrasound image, to generate an adaptive probability transformation function based on a probability distribution of the ultrasound image, to generate weights corresponding to the probability values by using the adaptive probability transformation function, and to measure an area or volume of a region of interest more accurately by using the weights.

In order to achieve the above-described technical objective, according to a first aspect of the present invention, there is provided an ultrasound-based bladder scanner system comprising: an ultrasound probe including an ultrasound transducer and a motor and configured to receive ultrasound signals for acquiring an ultrasound image; and at least one processor. The at least one processor is configured to generate the ultrasound image by using the ultrasound signals received from the ultrasound probe, to infer probability values indicating whether respective unit regions constituting the ultrasound image are included in a region of interest by using a pre-trained deep learning network, to convert the inferred probability values into weights for the respective unit regions by using a preset probability transformation function or an adaptive probability transformation function, and to extract information on the region of interest by using the weights.

In the ultrasound-based bladder scanner system according to the first aspect, it is preferable that the at least one processor is configured to, in order to extract information on the region of interest, multiply a unit area or a unit volume of each unit region constituting the ultrasound image by a corresponding weight of the unit region to obtain a weighted area or a weighted volume for each unit region, and to measure an area or a volume of the region of interest by summing the weighted areas or the weighted volumes of all unit regions constituting the ultrasound image.

In the ultrasound-based bladder scanner system according to the first aspect, it is preferable that the at least one processor is configured to extract statistical characteristics of a distribution of probability values for each ultrasound image, to determine one or more control parameters for the ultrasound image based on the extracted statistical characteristics, and to generate an adaptive probability transformation function corresponding to the ultrasound image by applying the control parameter to a preset probability transformation function.

In the ultrasound-based bladder scanner system according to the first aspect, it is preferable that the statistical characteristics of the distribution of the probability values include one or more of a mean, a variance, and a kurtosis of a histogram of the probability values of the ultrasound image.

In the ultrasound-based bladder scanner system according to the first aspect, it is preferable that the adaptive probability transformation function is an S-shaped linear or nonlinear function having continuity and differentiability over an entire input and output range.

In the ultrasound-based bladder scanner system according to the first aspect, it is preferable that the adaptive probability transformation function is configured to output a weight W(i,j) between 0 and 1 for a probability value p(i,j) corresponding to each unit region constituting the ultrasound image, the adaptive probability transformation function being implemented as a sigmoid function or a hyperbolic tangent (tanh) function having first and second control parameters, wherein the first control parameter controls a sensitivity of the probability transformation function and the second control parameter controls a center point of the probability transformation function.

In some embodiments, the adaptive probability transformation function is expressed by the following equation.

$$W ( i , j ) = \frac{1 + tanh a ( p ( i , j ) - b )}{2} = \frac{1}{1 + {e}^{- 2 a ( p ( i , j ) - b )}} \qquad 0 \leq p \leq 1$$

Here, a and b denote first and second control parameters, respectively, p(i,j) denotes a probability value of the pixel located at coordinates (i,j) of the ultrasound image, and W(i,j) denotes a weight assigned to the probability value p(i,j).

In the ultrasound-based bladder scanner system according to the first aspect, it is preferable that the preset probability transformation function assigns a preset minimum weight to probability values less than the first threshold value, assigns a preset maximum weight to probability values greater than the second threshold value, and assigns weights within the range between the minimum weight and the maximum weight to probability values between the first threshold value and the second threshold value in a linear or nonlinear manner. In some embodiments, the minimum weight is 0 and the maximum weight is 1.

In order to achieve the above-described technical objective, according to a second aspect of the present invention, there is provided a bladder volume measurement method performed by a processor included in a bladder scanner system, the method comprising: (a) receiving ultrasound signals from an ultrasound probe and generating an ultrasound image by using the received ultrasound signals; (b) inferring probability values indicating whether respective unit regions constituting the ultrasound image are included in a region of interest by using a pre-trained deep learning network; (c) converting the inferred probability values into weights for the respective unit regions by using a preset probability transformation function or an adaptive probability transformation function; and (d) extracting information on the region of interest by using the weights.

In the bladder volume measurement method according to the second aspect, it is preferable that step (d) comprises multiplying a unit area or a unit volume of each unit region constituting the ultrasound image by a corresponding weight of the unit region to obtain a weighted area or a weighted volume for each unit region, and measuring an area or a volume of the region of interest by summing the weighted areas or the weighted volumes of all unit regions constituting the ultrasound image.

In the bladder volume measurement method according to the second aspect, it is preferable that the method further comprises extracting statistical characteristics of a distribution of probability values corresponding to each ultrasound image, determining one or more control parameters for the ultrasound image based on the extracted statistical characteristics, and generating an adaptive probability transformation function corresponding to the ultrasound image by applying the control parameters to a preset probability transformation function.

In the bladder volume measurement method according to the second aspect, it is preferable that the statistical characteristics of the distribution of the probability values of the ultrasound image include statistical moments of the histogram, including one or more of a mean, a variance, and a kurtosis.

In the bladder volume measurement method according to the second aspect, it is preferable that the adaptive probability transformation function is an S-shaped linear or nonlinear function having continuity and differentiability over an entire input range.

In the bladder volume measurement method according to the second aspect, it is preferable that the adaptive probability transformation function is implemented as a sigmoid function or a hyperbolic tangent (tanh) function having first and second control parameters, the function being configured to output a weight W(i,j) between 0 and 1 for a probability value p(i,j) corresponding to each unit region constituting the ultrasound image, wherein the first control parameter adjusts a sensitivity of the probability transformation function and the second control parameter adjusts a center point of the probability transformation function.

In certain embodiments, the adaptive probability transformation function is expressed by the following equation.

$$W ( i , j ) = \frac{1 + tanh a ( p ( i , j ) - b )}{2} = \frac{1}{1 + {e}^{- 2 a ( p ( i , j ) - b )}} \qquad 0 \leq p \leq 1$$

Here, a and b denote first and second control parameters, respectively; p(i,j) denotes a probability value corresponding to a unit region (i,j) of the ultrasound image; and W(i,j) denotes a weight assigned to the probability value p(i,j).

In the bladder volume measurement method according to the second aspect, it is preferable that the preset probability transformation function assigns a preset minimum weight to probability values less than a first threshold value, assigns a preset maximum weight to probability values greater than a second threshold value, and assigns weights within the range between the minimum weight and the maximum weight to probability values between the first threshold value and the second threshold value in a linear or nonlinear manner. In some embodiments, the minimum weight is 0 and the maximum weight is 1.

According to the ultrasound-based bladder scanner system of the present invention, probability values corresponding to unit regions of an ultrasound image are extracted by using a region-of-interest extraction algorithm, weights corresponding to the probability values are generated by using an adaptive probability transformation function for each ultrasound image, and an area or a volume of a region of interest is calculated by using the weights. As a result, the ultrasound-based bladder scanner system according to the present invention is capable of calculating information such as an area or a volume of the region of interest with improved accuracy.

In addition, the ultrasound-based bladder scanner system according to the present invention analyzes a probability distribution corresponding to each ultrasound image, determines a sensitivity control parameter and a center-point control parameter based on the analyzed probability distribution information, and generates an adaptive probability transformation function optimized for each ultrasound image by using the determined control parameters. As a result, the ultrasound-based bladder scanner system according to the present invention is capable of further improving reliability of measurement results.

In addition, the ultrasound-based bladder scanner system according to the present invention measures an area or a volume of a region of interest by using probability values corresponding to all pixels constituting an ultrasound image, without performing binarization of the probability values or segmentation of the region of interest. As a result, the ultrasound-based bladder scanner system according to the present invention is capable of reducing sensitivity to threshold selection and improving accuracy of measurement.

### BRIEF DESCRIPTION OF THE DRAWINGS

FIG. 1 illustrates an example of an ultrasound-based bladder scanner system according to the related art.

FIG. 2 illustrates a two-dimensional ultrasound image acquired by using the bladder scanner system of FIG. 1.

In FIG. 3, (a) is a two-dimensional ultrasound image 810 acquired using a bladder scanner system, (b) is an image 820 generated from probability values output for each pixel of the two-dimensional ultrasound image 810 of (a) using a machine learning algorithm, (c) is an enlarged view of a portion 830 of (b), and (d) is a graph 840 showing the probability values corresponding to the portion 830 of (c).

FIG. 4A illustrates a two-dimensional ultrasound image. FIG. 4B illustrates an image reconstructed from probability values output by an extraction algorithm according to the related art. FIG. 4C illustrates an image obtained by binarizing the probability values with a threshold set to 0.5. FIG. 4D illustrates an image obtained by binarizing the probability values with a threshold set to 0.4.

FIG. 5 illustrates a graph showing a change in probability values according to scan depth along a single scan line in a bladder volume measurement system using an ultrasound scanner according to the related art.

FIG. 6 is a schematic configuration diagram illustrating an ultrasound-based bladder scanner system according to a preferred embodiment of the present invention.

FIG. 7 is a flowchart sequentially illustrating a method by which a controller measures an area or a volume of a bladder, which is a region of interest, in the ultrasound-based bladder scanner system according to the preferred embodiment of the present invention.

FIG. 8 is a graph illustrating an example of an adaptive probability transformation function in the ultrasound-based bladder scanner system according to the preferred embodiment of the present invention.

FIG. 9 is a graph illustrating an example of a preset probability transformation function in the ultrasound-based bladder scanner system according to the preferred embodiment of the present invention.

FIG. 10 is a geometric model illustrating an infinitesimal volume of a single voxel, which is a unit region in an ultrasound image expressed in a spherical coordinate system, in the ultrasound-based bladder scanner system according to the preferred embodiment of the present invention.

FIG. 11 is a geometric model illustrating an infinitesimal volume of a single voxel, which is a unit region in an ultrasound image expressed in a cylindrical coordinate system, in the ultrasound-based bladder scanner system according to the preferred embodiment of the present invention.

FIG. 12 is a chart comparing bladder volume measurements, errors, and error rates obtained using conventional methods and a method according to the preferred embodiment of the present invention in the ultrasound-based bladder scanner system.

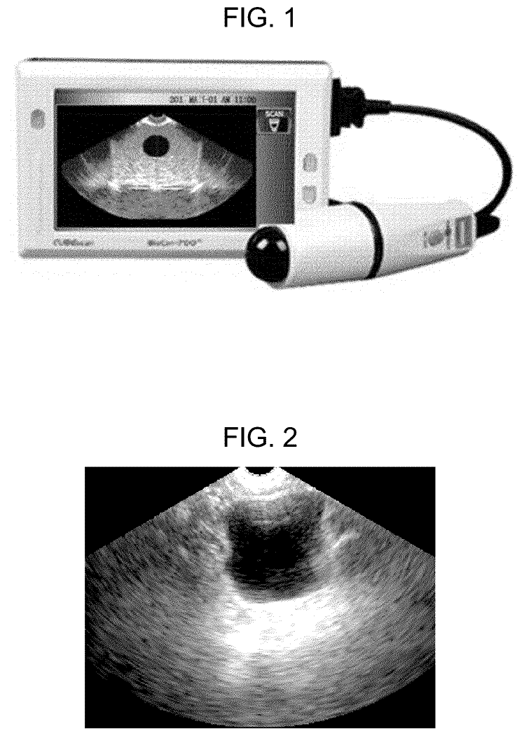

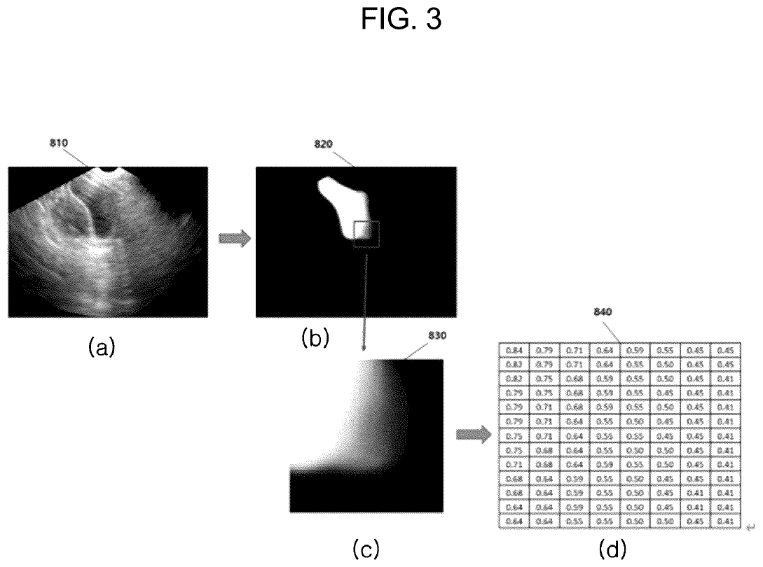

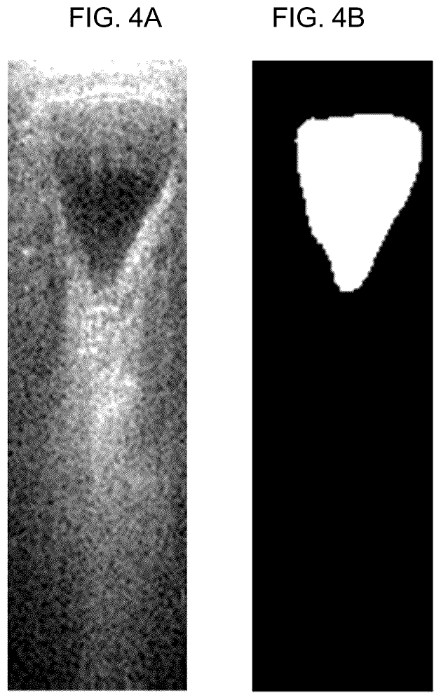

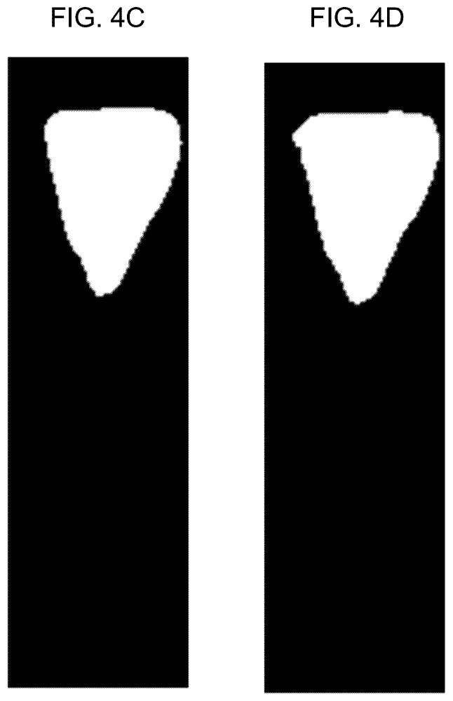

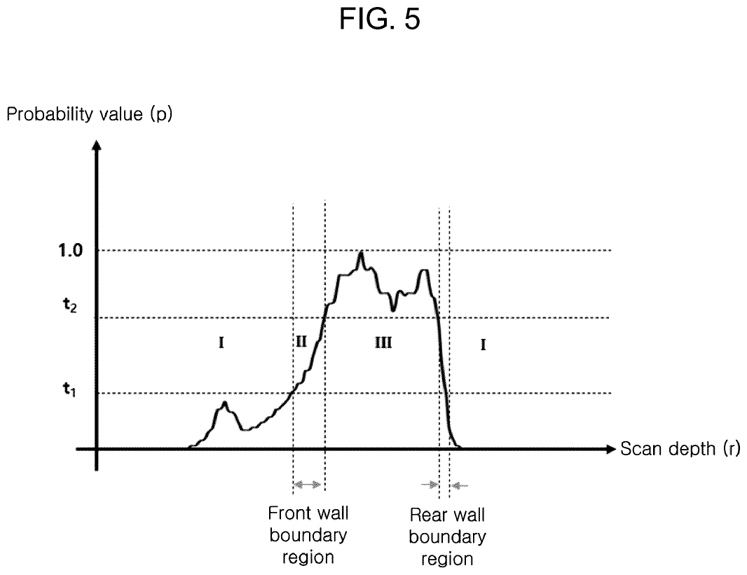

### DETAILED DESCRIPTION

Hereinafter, an ultrasound-based bladder scanner system according to a preferred embodiment of the present invention and a bladder volume measurement method performed by the system will be described in detail with reference to the accompanying drawings.

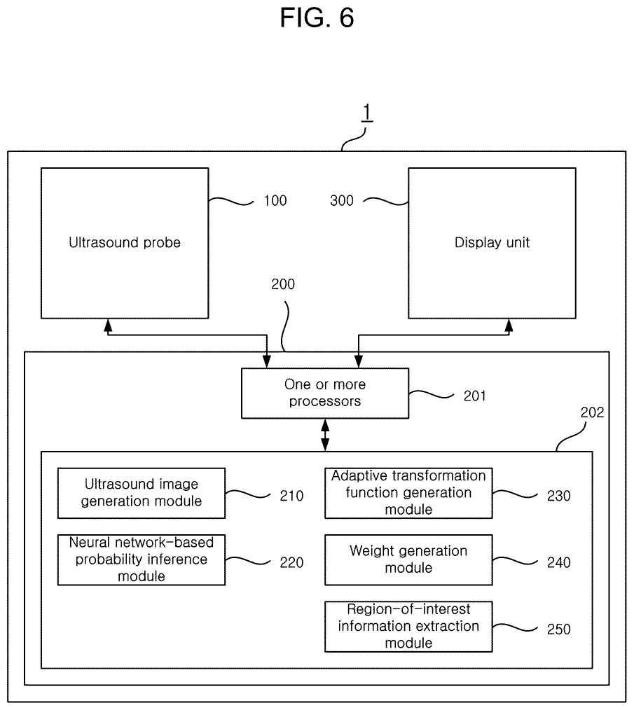

FIG. 6 is a schematic configuration diagram illustrating an ultrasound-based bladder scanner system according to the preferred embodiment of the present invention. Referring to FIG. 6, the ultrasound-based bladder scanner system 1 according to the preferred embodiment of the present invention includes an ultrasound probe 100, a controller 200, and a display unit 300. The controller 200 may include one or more processors 201 and a memory 202.

The ultrasound probe 100 includes an ultrasound transducer and is configured to transmit ultrasound waves to an object to be measured and to acquire ultrasound signals reflected from the object. As used herein, the term “object” refers to a physical subject including a human body or a portion thereof on which an ultrasound examination is performed. The ultrasound transducer may include a single element or a plurality of elements, and may transmit and receive ultrasound signals for a bladder region to provide basic data for generating a two-dimensional or three-dimensional ultrasound image.

In addition, the ultrasound probe 100 may further include a motor. The motor of the ultrasound probe may be configured to mechanically rotate the ultrasound transducer under control of the controller 200. By rotation of the motor, the ultrasound probe may be configured to sequentially acquire ultrasound signals at different angles.

The controller 200 may include one or more processors 201 and a memory 202. The memory 202 may store programs composed of instructions executable by the one or more processors 201 of the controller 200. The programs stored in the memory 202 may include an ultrasound image generation module 210, a neural network-based probability inference module 220, an adaptive transformation function generation module 230, a weight generation module 240, and a region-of-interest information extraction module 250. By executing the programs stored in the memory 202, the processor 201 of the controller 200 may be configured to generate an ultrasound image based on ultrasound signals received from the ultrasound probe 100 and to extract information regarding a region of interest from the ultrasound image.

As used herein, the term “module” refers to software, firmware, hardware, or any combination thereof implemented by one or more processors.

Meanwhile, the programs stored in the memory 202 may further include a motor control module configured to control rotation of the transducer by controlling the motor of the ultrasound probe 100, and an output module configured to control display of images or information on the display unit 300.

The display unit 300 may include a display panel such as a liquid crystal display (LCD), an organic light-emitting diode (OLED) display, or a similar display device. The display unit 300 may be configured to visually output an ultrasound image or information regarding a region of interest under control of the controller 200. The display unit 300 may output the ultrasound image, positional information, shape information, boundary indications of a bladder region, or numerical information related to a bladder volume provided from the controller 200. In addition, the display unit 300 may be configured to display the region of interest in an overlay form on the ultrasound image, or to provide analysis results regarding the region of interest in a text or graphic form.

Meanwhile, the ultrasound image generation module 210, the neural network-based probability inference module 220, the adaptive transformation function generation module 230, the weight generation module 240, and the region-of-interest information extraction module 250 of the controller 200 may be implemented as software executable by the processor of the controller 200 and stored in the memory 202, or may be implemented in hardware or firmware. Hereinafter, the modules stored in the memory 202 of the controller 200 and executed by the processor 201 will be described in detail.

The ultrasound image generation module 210 is configured to generate an ultrasound image by using ultrasound signals received from the ultrasound probe 100. More specifically, the ultrasound image generation module 210 may receive ultrasound echo signals acquired by the ultrasound transducer, convert the received ultrasound echo signals into digital signals, and perform at least one signal processing operation on the digital signals, including signal alignment, amplification, filtering, or interpolation.

In addition, the ultrasound image generation module 210 may be configured to process and combine a plurality of ultrasound signals acquired at different angles or positions to generate a two-dimensional ultrasound image or a three-dimensional ultrasound image. In this case, the ultrasound image generation module 210 may be configured to calculate an image value corresponding to each pixel or voxel of the ultrasound image by using at least one of time delay, signal intensity, or phase information of the received ultrasound signals.

According to one embodiment, the ultrasound image generation module 210 may be configured to generate a plurality of two-dimensional ultrasound images based on ultrasound signals sequentially acquired in accordance with mechanical rotation of the ultrasound probe 100 by the motor or manual rotation by a user, and to reconstruct the plurality of two-dimensional ultrasound images to generate a three-dimensional ultrasound image.

The neural network-based probability inference module 220 may include a pre-trained deep learning neural network and may be configured to infer and provide, for each unit region constituting the ultrasound image, a probability value indicating whether the unit region is included in the region of interest. The pre-trained deep learning neural network may be implemented as a neural network model that has been trained in advance using a plurality of training data sets. The training data may include ultrasound image data in which a bladder region has been labeled.

More specifically, the neural network-based probability inference module 220 may receive, as an input, the ultrasound image generated by the ultrasound image generation module 210, and may evaluate a likelihood that each unit region is included in a region of interest by dividing the ultrasound image into a plurality of unit regions. As used herein, the term “unit region” refers to a minimum processing unit defined according to a spatial resolution of the ultrasound image. For example, in the case of a two-dimensional ultrasound image, the unit region may be a single pixel or a pixel block composed of a plurality of pixels (e.g., an N×N block), and in the case of a three-dimensional ultrasound image, the unit region may be a voxel or a voxel block composed of a plurality of voxels. In addition, the unit region may be defined as a grid-based region identified according to row and column coordinates (and depth coordinates, in the case of a three-dimensional image) of the ultrasound image.

The deep learning network of the neural network-based probability inference module 220 may employ a convolutional neural network (CNN) or a neural network having a U-Net architecture, which is a variation of a CNN, and various other types of neural network architectures may also be selected and used. A neural network having the U-Net architecture includes an encoder and a decoder, and outputs, for each unit region of an input image, a probability of belonging to a region of interest or a class label. The encoder includes a plurality of convolution layers and pooling layers, and extracts feature maps while transforming the input image into a lower resolution representation. The decoder restores the extracted low-resolution feature maps to a resolution corresponding to that of the original input image. The neural network having the U-Net architecture is widely used in the field of medical imaging to segment organs or lesion regions. Because the U-Net architecture generally has fewer parameters compared to other deep learning models, it provides advantages in terms of faster training speed and high accuracy. In addition, the U-Net architecture is suitable for incremental learning or fine-tuning to improve performance of the network model when new input data are added, thereby facilitating reuse of a previously trained model to further enhance performance.

The neural network-based probability inference module 220 inputs the ultrasound image into the deep learning network, and the deep learning network may be configured to infer and output, for each unit region of the ultrasound image, a probability or likelihood value indicating whether the unit region is included in a region of interest. For example, the probability value may be expressed as a real number within a range of 0 to 1, and a higher probability value indicates a higher likelihood that the corresponding unit region is included in the region of interest.

According to one embodiment, the neural network-based probability inference module 220 may be configured to generate, as an output of the deep learning network, a set of probability values in the form of a probability map. That is, the probability map is a map in which probability values indicating inclusion in a region of interest are mapped to all unit regions of the ultrasound image.

In addition, the neural network-based probability inference module 220 may be configured to normalize or scale the input image in consideration of various quality variations of the ultrasound image before providing the input image to the deep learning network. Here, quality variations of the ultrasound image may result from noise, changes in reflection intensity, patient body shape, or variations in measurement posture. Accordingly, the neural network-based probability inference module 220 may stably infer and provide the probability map, which is a set of probability values indicating inclusion in the region of interest, even for ultrasound images acquired under various conditions.

The adaptive transformation function generation module 230 may be implemented as a program executable by the processor of the controller 200 and stored in the memory 202. The adaptive transformation function generation module 230 determines first and second control parameters optimized for each ultrasound image based on statistical characteristics of a probability distribution of the corresponding ultrasound image, and provides the determined control parameters to the weight generation module 240. The adaptive transformation function generation module 230 may be configured to determine the first and second control parameters of a probability transformation function by using an algorithm that automatically calculates the control parameters based on statistical distribution characteristics of the probability map of the ultrasound image. The statistical distribution characteristics of the probability map used to determine the first and second control parameters may include at least one of a mean, a variance, or a kurtosis of a histogram distribution of the ultrasound image. The weight generation module 240 may generate an adaptive probability transformation function by applying the control parameters determined by the adaptive transformation function generation module 230 to the probability transformation function.

The weight generation module 240 may be configured to convert the probability value of each unit region inferred by the neural network-based probability inference module 220 into a weight by using an adaptive probability transformation function or a preset probability transformation function. Hereinafter, the adaptive probability transformation function and the preset probability transformation function used in the present invention will be described in detail.

The adaptive probability transformation function that may be used in the weight generation module 240 may be configured as an S-shaped linear or non-linear function, and is characterized in that both input values and output values have continuity and differentiability over an entire domain. The adaptive probability transformation function may be generated in an optimized manner according to each ultrasound image.

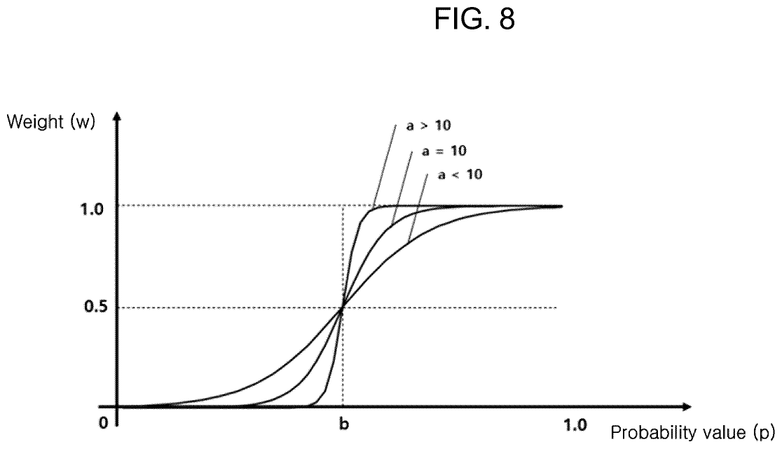

FIG. 8 illustrates an example of the adaptive probability transformation function in the ultrasound-based bladder scanner system according to the preferred embodiment of the present invention. The example of the adaptive probability transformation function shown in FIG. 8 may be represented by the following equation.

$$W ( i , j ) = \frac{1 + tanh a ( p ( i , j ) - b )}{2} = \frac{1}{1 + {e}^{- 2 a ( p ( i , j ) - b )}} \qquad 0 \leq p \leq 1$$

The adaptive probability transformation function according to the above equation may be configured as a sigmoid function or a hyperbolic tangent (tanh) function having a first control parameter a and a second control parameter b with respect to a probability value p(i,j) corresponding to each unit region of the ultrasound image, and may output a weight W(i,j) having a value between 0 and 1.

The first control parameter a is a factor that determines a sensitivity or slope of the adaptive probability transformation function. When the value of a is relatively small, the slope of the transformation function becomes gradual, such that information corresponding to an intermediate probability range is progressively reflected in a volume calculation process. Conversely, when the value of a is relatively large, the slope of the transformation function becomes steep, such that a weight of high-confidence pixels rapidly converges to 1. However, when the value of a is excessively large, an output of the probability transformation function may become substantially binarized, resulting in loss of continuous probability information, and thereby increasing a likelihood of error in an area or volume calculation process. Accordingly, it is preferable that the first control parameter a be set within a range of less than 30.

Meanwhile, the second control parameter b is a factor that determines a reference point or center point of the adaptive probability transformation function, and may be configured to control a position of an inflection point at which a weight value becomes 0.5 on the probability map.

In one example of the system according to the present invention, the first and second control parameters a and b of the adaptive probability transformation function may be preset through experimental analysis in which a calculated bladder volume is compared with an actual voided volume such that a volume error is minimized. In another example of the system according to the present invention, through the adaptive transformation function generation module 230, one or both of the first and second control parameters of the adaptive probability transformation function may be adaptively and automatically determined for the corresponding ultrasound image based on statistical distribution characteristics of the probability map of the ultrasound image.

Accordingly, even when a shape of a probability value distribution for the ultrasound images varies due to a noise level or contrast variation of the ultrasound images, the control parameters of the adaptive probability transformation function may be automatically calculated and reflected in real time by using the adaptive transformation function generation module 230. As a result, a sensitivity of the adaptive probability transformation function may be adjusted according to characteristics of each ultrasound image, thereby improving consistency and reliability of volume estimation.

The algorithm used for automatically calculating the control parameters in the adaptive transformation function generation module 230 may include statistical analysis techniques such as adaptive thresholding, Otsu's thresholding, and Yen's thresholding. In the present invention, it may be preferable to select a method that minimizes a volume estimation error among these techniques.

Among the above-described analysis techniques, Otsu's thresholding algorithm may be represented by the following equations.

$${σ}_{B}^{2} ( t ) = {ω}_{0} ( t ) {( {μ}_{0} ( t ) - {μ}_{T} )}^{2} + {ω}_{1} ( t ) {( {μ}_{1} ( t ) - {μ}_{T} )}^{2}$$

$${t}_{- Otsu} = \begin{matrix}arg max \\ t\end{matrix} {σ}_{B}^{2} ( t )$$

Here, the inter-class variance

$${σ}_{B}^{2} ( t )$$

represents a variance between classes when the ultrasound image is divided into two classes (for example, a region of interest and a non-region of interest) based on a threshold. ω₀(t) denotes a probability of class 0 corresponding to values less than or equal to the threshold t, ω₁(t) denotes a probability of class 1 corresponding to values greater than the threshold t, μ₀(t) denotes a mean value of class 0, μ₁(t) denotes a mean value of class 1, and μ_{T} denotes a mean value of all data. According to the Otsu threshold determination method based on the above equations, the threshold tₒₜₛᵤ that maximizes the inter-class variance

$${σ}_{B}^{2} ( t )$$

may be automatically calculated by analyzing a pixel value distribution of the probability map.

In the present invention, the threshold tₒₜₛᵤ may be applied as the second control parameter b of the adaptive probability transformation function, thereby adaptively adjusting an inflection point of the probability transformation function to a statistical center of each image. Furthermore, the calculated maximum inter-class variance

$${σ}_{B}^{2} ( t )$$

may be used as a statistical indicator representing contrast and uncertainty levels of the ultrasound image. In the present invention, the inter-class variance

$${σ}_{B}^{2} ( t )$$

may also be applied to determination of the first control parameter a that determines sensitivity of the probability transformation function. That is, when the inter-class variance

$${σ}_{B}^{2} ( t )$$

is high, the first control parameter a may be set to a relatively high value such that a weight for high-probability pixels increases rapidly, and when the inter-class variance

$${σ}_{B}^{2} ( t )$$

is low, the first control parameter a may be set to a relatively low value such that information in an intermediate probability range is smoothly reflected in a volume calculation process.

An optimal value of the first control parameter a may be determined through a look-up table (LUT) experimentally mapped to minimize a volume estimation error (RMSE) for respective ranges of inter-class variance

$${σ}_{B}^{2} ( t )$$

based on a plurality of training data sets. Accordingly, the adaptive transformation function generation module 230 may further include a look-up table for setting the first control parameter. The look-up table for setting the first control parameter a may include values of inter-class variance

$${σ}_{B}^{2} ( t )$$

and corresponding values of the first control parameter that minimize the volume estimation error (RMSE). Therefore, the adaptive transformation function generation module 230 may retrieve, from the LUT, the first control parameter a value of a corresponding to the inter-class variance

$${σ}_{B}^{2} ( t )$$

calculated in real time for the ultrasound image, and apply the retrieved value to the probability transformation function.

As described above, by adaptively determining the first and second control parameters of the probability transformation function based on the inter-class variance

$${σ}_{B}^{2} ( t ) ,$$

the system according to the present invention may calculate a volume while maintaining continuity of probability values without performing classification or boundary extraction of the probability values, even when image quality, contrast, noise, or artifacts vary. As a result, the system according to the present invention may improve accuracy and consistency of volume estimation, as well as robustness against variations in ultrasound imaging conditions.

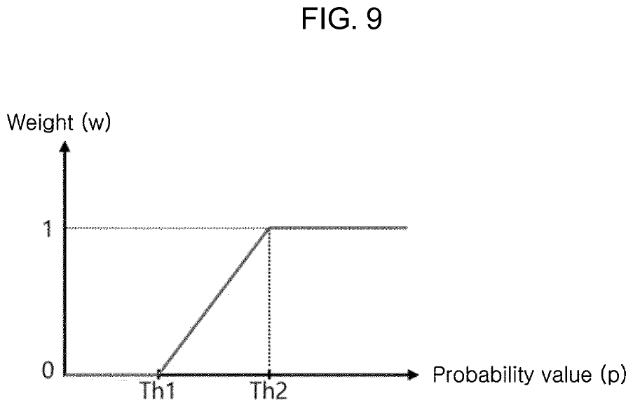

FIG. 9 illustrates an example of the preset probability transformation function in the bladder scanner system according to the preferred embodiment of the present invention. Referring to FIG. 9, the preset probability transformation function that may be used in the weight generation module 240 according to the present invention includes a first threshold Th₁ and a second threshold Th₂. The preset probability transformation function assigns a preset minimum weight to a probability value p less than the first threshold Th₁, assigns a preset maximum weight to a probability value p greater than the second threshold Th₂, and assigns a weight between the minimum weight and the maximum weight, either linearly or non-linearly, to a probability value p between the first threshold Th₁ and the second threshold Th₂. The minimum weight may be set to 0, and the maximum weight may be set to 1.

Here, the first threshold Th₁ and the second threshold Th₂ may be determined in consideration of characteristics of the system including noise and artifacts. Pixels having probability values less than or equal to the first threshold Th₁ may be regarded as belonging to a background region, and pixels having probability values greater than or equal to the second threshold Th2 may be regarded as belonging to a region of interest.

An example of the above-described probability transformation function may be expressed by the following equation.

$$p < {Th}_{1} : w = 0$$

$${Th}_{1} \leq p \leq {Th}_{2} : w = ( p - {Th}_{1} ) / ( {Th}_{2} - {Th}_{1} )$$

$$p > {Th}_{2} : w = 1$$

In the above equation, the weight has been calculated using a first-order linear expression; however, this is merely provided as an illustrative example. Accordingly, in consideration of system performance, various modified forms of mathematical expressions other than the first-order linear expression may also be used to calculate the weight.

The first threshold Th₁ and the second threshold Th₂ set in the present embodiment may affect calculation of an area or volume of the region of interest. However, compared to conventional techniques that separate a background region and a region of interest by a binarization method, the method according to the present embodiment exhibits significantly lower sensitivity to the threshold values. This is because, in calculating the area or volume of the region of interest, the bladder scanner system according to the present embodiment applies weights to probability values of unit regions located at a boundary between the background region and the region of interest, taking into account, for example, a distance from a center of the region of interest. Therefore, even if the first threshold Th₁ and the second threshold Th₂ of the probability transformation function are slightly changed in the bladder scanner system according to the present embodiment, a final result does not change significantly.

The region-of-interest information extraction module 250 may extract information regarding the region of interest based on weights corresponding to unit regions constituting the ultrasound image. The region-of-interest information extraction module 250 may be configured to multiply a unit area or a unit volume of each unit region constituting the ultrasound image by a corresponding weight to obtain a weighted area or a weighted volume for each unit region, and to calculate an area or a volume of the region of interest by summing weighted areas or weighted volumes of all unit regions constituting the ultrasound image.

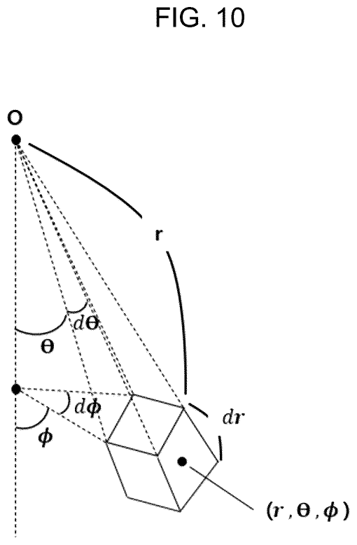

The ultrasound image may be represented using a spherical coordinate system (r, θ, φ) having a rotation center of the transducer as an origin. FIG. 10 illustrates a geometric model of an infinitesimal volume of a single voxel, which is a unit region in an ultrasound image represented in the spherical coordinate system, in the ultrasound-based bladder scanner system according to the preferred embodiment of the present invention. Referring to FIG. 10, an infinitesimal volume of a voxel located at a point (r, θ, φ) in the spherical coordinate system is illustrated. In an ultrasound image represented in the spherical coordinate system, an infinitesimal volume dV represented by a single voxel may be expressed as follows.

$$dV = {r}^{2} sin θ d θ d φdr$$

Here, dθ represents an angular interval between adjacent scan lines, dφ represents an angular interval between adjacent scan planes, and dr represents a distance interval between adjacent sampling points along a single scan line.

By applying the above-described weights, a volume may be calculated using the following equation.

$$V = {\int }_{{r}_{min}}^{{r}_{max}} {\int }_{0}^{π} {\int }_{\frac{- {θ}_{max}}{2}}^{\frac{{θ}_{max}}{2}} W ( r , θ , φ ) {r}^{2} sin θ d θ d φdr$$

Here, W(r, θ, φ) denotes a weight calculated for a pixel located at a point (r, θ, φ).

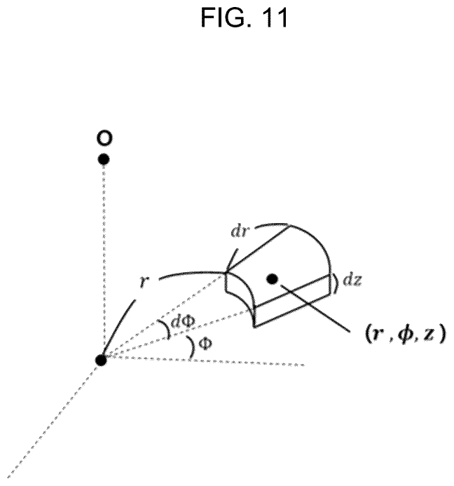

Meanwhile, the ultrasound image may be represented using a cylindrical coordinate system (r, Φ, z) based on a rotational central axis of the transducer. FIG. 11 illustrates a geometric model of an infinitesimal volume of a single voxel, which is a unit region in an ultrasound image represented in the cylindrical coordinate system, in the ultrasound-based bladder scanner system according to a preferred embodiment of the present invention. In an ultrasound image represented in the cylindrical coordinate system, an infinitesimal volume dV represented by a single voxel may be expressed by the following equation.

$$dV = rdrd Φ dz$$

Here, dr represents a horizontal length occupied by a single pixel, dΦ represents an angular interval between adjacent scan planes, and dz represents a vertical length occupied by a single pixel.

By applying the above-described weights, a volume V may be calculated using the following equation.

$$V = {\int }_{0}^{{r}_{max}} {\int }_{0}^{2 π} {\int }_{{z}_{min}}^{{z}_{max}} W ( r , Φ , z ) r drd Φ dz$$

Here, W(r, Φ, z) denotes a weight calculated for a pixel located at a point (r, Φ, z).

As described above, the bladder scanner system according to the present invention is characterized in that a volume is calculated by using only weight information obtained by transforming probability values inferred by deep learning, without segmenting the bladder region from the ultrasound image. Accordingly, the bladder scanner system according to the present invention may probabilistically estimate a volume of the bladder, which is the region of interest.

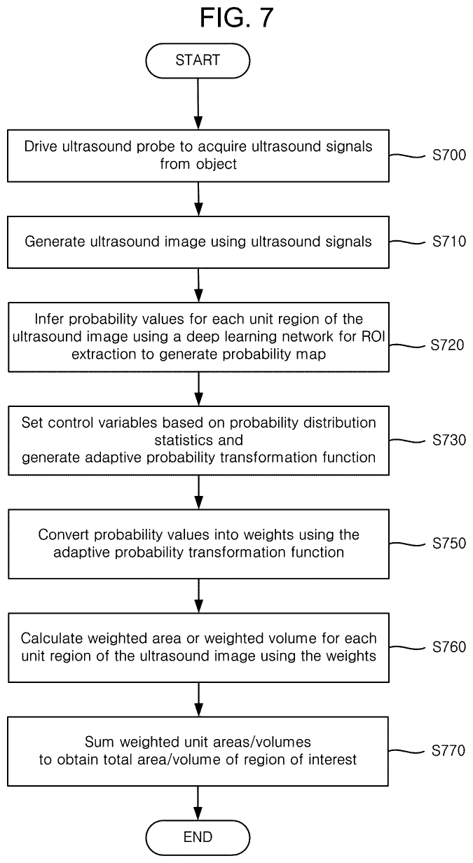

Hereinafter, a method by which the controller of the ultrasound-based bladder scanner system according to the preferred embodiment of the present invention calculates a bladder volume will be described in more detail. FIG. 7 is a flowchart sequentially illustrating a method of measuring an area or a volume of a bladder, which is a region of interest, by the controller in the ultrasound-based bladder scanner system according to the preferred embodiment of the present invention.

Referring to FIG. 7, the controller of the bladder scanner system according to the present invention first drives the ultrasound probe to acquire ultrasound signals for an object (step 700). Next, the controller generates an ultrasound image using the ultrasound image generation module based on the ultrasound signals received from the ultrasound probe (step 710).

Subsequently, the controller uses the neural network-based probability inference module including a pre-trained deep learning network to infer, for each unit region constituting the ultrasound image, a probability value indicating whether the unit region is included in a region of interest, thereby acquiring a probability map for the ultrasound image (step 720).

Next, the controller may use the adaptive transformation function generation module to determine control parameters of a probability transformation function optimized for each ultrasound image based on statistical characteristics of a probability distribution of each ultrasound image, thereby generating an adaptive probability transformation function optimized for each ultrasound image (step 730). The statistical characteristics of the probability distribution used to determine the control parameters may include at least one or more of a mean, a variance, or a kurtosis of a histogram of probability values of the ultrasound image.

Next, the controller may use the weight generation module to convert probability values of unit regions inferred by the neural network-based probability inference module into weights by applying the adaptive probability transformation function for each ultrasound image or a preset probability transformation function, thereby obtaining a weight map for the ultrasound image (step 750).

Next, the controller may use the region-of-interest information extraction module to multiply a unit area or a unit volume of each unit region constituting the ultrasound image by a corresponding weight to obtain a weighted area or a weighted volume for each unit region (step 760). Thereafter, the controller may calculate and provide an area or a volume of the region of interest by summing weighted areas or weighted volumes of all unit regions constituting the ultrasound image (step 770).

A performance comparison was conducted between the weight-based volume calculation method according to the present invention and a conventional binarization-based volume calculation method. The comparative experiment was performed on ultrasound images under three different conditions, including:
(i) an ultrasound image shown in FIG. 8 (case C);
(ii) an image exhibiting relatively high accuracy at a threshold of 0.4 (case A); and
(iii) an image exhibiting highest accuracy at a threshold of 0.5 (case B).

In the conventional binarization-based method, a threshold value was fixed at 0.4 or 0.5 for a probability map, binarization was performed based on the fixed threshold value, and a bladder volume was calculated using a generated binary mask. In contrast, in the method according to the present invention, weights were calculated for each cross-sectional ultrasound image using an adaptive probability transformation function, and a bladder volume was calculated based on the calculated weights. More specifically, a hyperbolic tangent (tanh) transformation function was applied as the adaptive probability transformation function, and control parameters a and b of the adaptive probability transformation function were configured to be automatically calculated for each cross-sectional ultrasound image using an Otsu thresholding algorithm. Through this configuration, an adaptive probability transformation function corresponding to probability distribution characteristics of each image may be generated.

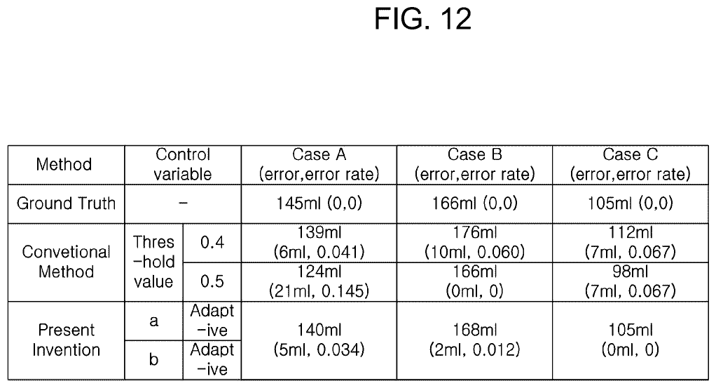

FIG. 12 is a table illustrating a comparison of bladder volumes, volume errors, and error rates calculated by applying conventional volume calculation methods and the volume calculation method according to the present invention in the ultrasound-based bladder scanner system according to the preferred embodiment of the present invention. Referring to FIG. 12, the following differences were identified based on the above-described experimental results.

In the conventional binarization-based method, when a threshold value of 0.4 was fixed and applied to binarize the probability map, a maximum volume measurement error of up to 6.7% occurred. In addition, when a threshold value of 0.5 was fixed and applied for binarization, a maximum measurement error of up to 14.5% occurred. This indicates that a volume calculation result may vary sensitively depending on a selection of a fixed threshold value.

In contrast, when the weight-based volume calculation method according to the present invention was applied, a maximum measurement error was maintained within 3.4% for all experimental cases. This demonstrates that, unlike the conventional binarization method dependent on a specific threshold value, the present invention enables stable volume calculation even under variations in imaging conditions by adaptively responding to probability distribution characteristics. Accordingly, compared to the fixed-threshold-based volume calculation method, the present invention improves volume calculation accuracy, enhances error stability with respect to changes in imaging conditions, and significantly improves consistency and reliability of volume estimation results.

Although the present invention has been described above with reference to preferred embodiments thereof, the embodiments are provided for illustrative purposes only and do not limit the present invention. Those skilled in the art will appreciate that various modifications and applications may be made without departing from the essential characteristics of the present invention. Accordingly, such modifications and applications should be construed as falling within the scope of the present invention as defined by the appended claims.

What is claimed is:

1. An ultrasound-based bladder scanner system comprising:
an ultrasound probe including an ultrasound transducer and a motor, the ultrasound probe being configured to receive ultrasound signals for acquiring an ultrasound image; and
at least one processor,
wherein the at least one processor is configured to:
generate the ultrasound image using the ultrasound signals received from the ultrasound probe;
infer, using a pre-trained deep learning network, for each unit region constituting the ultrasound image, a probability value indicating whether the unit region is included in a region of interest;
convert the inferred probability values for the unit regions into weights using a preset probability transformation function or an adaptive probability transformation function; and
extract information regarding the region of interest using the weights.

2. The ultrasound-based bladder scanner system of claim 1, wherein the at least one processor is configured to, in order to extract the information regarding the region of interest,
multiply a unit area or a unit volume of each unit region constituting the ultrasound image by a corresponding weight to obtain a weighted area or a weighted volume for each unit region, and
calculate a total area or a total volume of the region of interest by summing weighted areas or weighted volumes of all unit regions constituting the ultrasound image.

3. The ultrasound-based bladder scanner system of claim 1, wherein the at least one processor is configured to:
extract statistical characteristics of a distribution of probability values for each ultrasound image;
determine at least one control parameter for the corresponding ultrasound image based on the extracted statistical characteristics; and
generate an adaptive probability transformation function corresponding to each ultrasound image by applying the control parameter to a probability transformation function.

4. The ultrasound-based bladder scanner system of claim 3, wherein the statistical characteristics of the distribution of probability values include one or more of a mean, a variance, or a kurtosis of a histogram of the probability values of the ultrasound image.

5. The ultrasound-based bladder scanner system of claim 1, wherein the adaptive probability transformation function is an S-shaped linear or non-linear function, and wherein input values and output values of the adaptive probability transformation function are continuous and differentiable over an entire domain.

6. The ultrasound-based bladder scanner system of claim 1, wherein the adaptive probability transformation function comprises a sigmoid function or a hyperbolic tangent (tanh) function having a first control parameter and a second control parameter with respect to a probability value p(i,j) corresponding to each unit region of the ultrasound image, and outputs a weight W(i,j) having a value between 0 and 1,
wherein the first control parameter controls a sensitivity of the probability transformation function, and
wherein the second control parameter controls a center point of the probability transformation function.

7. The ultrasound-based bladder scanner system of claim 1, wherein the adaptive probability transformation function is represented by the following equation:
$$W ( i , j ) = \frac{1 + tanh a ( p ( i , j ) - b )}{2} = \frac{1}{1 + {e}^{- 2 a ( p ( i , j ) - b )}} 0 \leq p \leq 1$$
wherein a and b are a first control parameter and a second control parameter, respectively, p(i,j) denotes a probability value corresponding to a pixel (i,j), and W(i,j) denotes a weight of p(i,j).

8. The ultrasound-based bladder scanner system of claim 1, wherein the preset probability transformation function assigns a preset minimum weight to a probability value less than a first threshold, assigns a preset maximum weight to a probability value greater than a second threshold, and assigns a weight within a range between the minimum weight and the maximum weight, either linearly or non-linearly, to a probability value between the first threshold and the second threshold.

9. The ultrasound-based bladder scanner system of claim 8, wherein the minimum weight is 0 and the maximum weight is 1.

10. A bladder volume measurement method performed by a processor of a ultrasound-based bladder scanner system configured to measure and provide a bladder volume using ultrasound signals, the method comprising:
(a) receiving ultrasound signals from an ultrasound probe and generating an ultrasound image using the received ultrasound signals;
(b) inferring, using a pre-trained deep learning network, for each unit region constituting the ultrasound image, a probability value indicating whether the unit region is included in a region of interest;
(c) converting the inferred probability values for the unit regions into weights using a preset probability transformation function or an adaptive probability transformation function; and
(d) extracting information regarding the region of interest using the weights.

11. The bladder volume measurement method of claim 10, wherein step (d) comprises:
(d1) multiplying a unit area or a unit volume of each unit region constituting the ultrasound image by a corresponding weight to obtain a weighted area or a weighted volume for each unit region; and
(d2) measuring an area or a volume of the region of interest by summing weighted areas or weighted volumes of all unit regions constituting the ultrasound image.

12. The bladder volume measurement method of claim 10, further comprising:
extracting statistical characteristics of a distribution of probability values for each ultrasound image;
determining a control parameter for the corresponding ultrasound image based on the extracted statistical characteristics; and
generating the adaptive probability transformation function corresponding to each ultrasound image by applying the control parameter to a probability transformation function.

13. The bladder volume measurement method of claim 12, wherein the statistical characteristics of the distribution of probability values include one or more of a mean, a variance, or a kurtosis of a histogram of the probability values of the ultrasound image.

14. The bladder volume measurement method of claim 10, wherein the adaptive probability transformation function is an S-shaped linear or non-linear function, and wherein input values and output values of the adaptive probability transformation function are continuous and differentiable over an entire domain.

15. The bladder volume measurement method of claim 10, wherein the adaptive probability transformation function comprises a sigmoid function or a hyperbolic tangent (tanh) function having a first control parameter and a second control parameter with respect to a probability value p(i,j) corresponding to each unit region of the ultrasound image, and outputs a weight W(i,j) having a value between 0 and 1,
wherein the first control parameter controls a sensitivity of the probability transformation function, and
wherein the second control parameter controls a center point of the probability transformation function.

16. The bladder volume measurement method of claim 10, wherein the adaptive probability transformation function is represented by the following equation:
$$W ( i , j ) = \frac{1 + tanh a ( p ( i , j ) - b )}{2} = \frac{1}{1 + {e}^{- 2 a ( p ( i , j ) - b )}} 0 \leq p \leq 1$$
wherein a and b are a first control parameter and a second control parameter, respectively, p(i,j) denotes a probability value corresponding to a pixel (i,j), and W(i,j) denotes a weight of p(i,j).

17. The bladder volume measurement method of claim 10, wherein the preset probability transformation function assigns a preset minimum weight to a probability value less than a first threshold, assigns a preset maximum weight to a probability value greater than a second threshold, and assigns a weight within a range between the minimum weight and the maximum weight, either linearly or non-linearly, to a probability value between the first threshold and the second threshold.

18. The bladder volume measurement method of claim 17, wherein the minimum weight is 0 and the maximum weight is 1.
